(19) United States
(12) Patent Application Publication
(10) Pub. No.: US 2006/0264932 A1
(43) Pub. Date: Nov. 23, 2006

(54) Attachment to bone

(76) Inventor: Jeffrey K. Bert, Coos Bay, OR (US)

Correspondence Address: CHERNOFF, VILHAUER, MCCLUNG & STENZEL, 1600 ODS TOWER, 601 SW SECOND AVENUE, PORTLAND, OR, 97204-3157, US

(21) Appl. No.: 11/123,812

(22) Filed: May 6, 2005

(51) Int. Cl. A61B 17/56

(52) U.S. Cl. 606/60

(57) ABSTRACT

A bone-engaging attachment device including a shaft threaded or otherwise adapted to mate with or to accept a fastener, and having a head extending radially at one end of the shaft. A method of attachment of the device to a bone includes preparing a hole in the bone large enough to receive the head and a channel extending from the hole, inserting the head into the hole, and moving the device to a location where the shaft extends outward through the channel while the head is engaged with the bone.

### BACKGROUND OF THE INVENTION

The present invention relates to bone-engaging fixation devices used in orthopedic surgery, and particularly to devices and a method for their use for positioning, immobilization, or reattachment of bones and attachment of other tissues to bones.

Orthopedic surgeons use bone-engaging fixation devices for a wide variety of purposes. Soft tissue repairs often require reattachment of the soft tissue to a bone. In particular, when surgically repairing an injured joint, damaged soft tissues such as ligaments and tendons are often reattached to bone.

When surgeons reduce fractures or reposition bones, rods or cables are often attached to fixation devices implanted in different bones or regions of a bone. During spinal repair surgery, rods or wires are often used to interconnect vertebrae in order to accomplish spinal fusions.

Fixation devices are often implanted into holes drilled into a bone. A small hole is typically drilled through the outer, cortex, layer of the bone and into the inner, cancellous, part of the bone, and various types of securing features such as resilient barbs on pins, screw threads, or grooved surfaces on pins or staples, are used to secure such fixation devices to the bone.

As a fixation device performs its function, bodily tissues or devices attached to the fixation device exert stress and the fixation device must be implanted securely enough to resist such stress and remain attached to the bone. If the fixation device does not have enough pull-out resistance to remain adequately securely attached to the bone, especially in the case of weakened or osteoporotic bone, a surgical repair may be compromised or fail. In certain bones, such as the occipital portion of the skull, it may be desired to attach an appliance securely in a location where stress may be applied in an outward direction, tending to withdraw the fixation device, and with great enough force to present a risk of withdrawing a fixation device of previous known designs.

What is desired, then, is a bone-engaging fixation device with ample pull-out resistance, and that can reliably withstand greater forces in at least some directions than previously available fixation devices can bear, and a method for attachment to bone utilizing such a device.

### SUMMARY OF THE INVENTION

The present invention answers the needs mentioned above, by providing a bone-engaging fixation device for use in orthopedic surgery that includes a shaft and a radially extending head at one end of the shaft. The fixation device may be implanted in a bone by forming a generally keyhole-shaped opening through the cortex layer of the bone, and thereafter inserting the head of the fixation device inward through the larger part of the hole. The device is then pushed laterally, with its head adjacent the inner boundary of the cortex layer, so that the shaft of the device is in a narrow channel part of the keyhole-shaped opening, extending outward from within the bone. The shaft thus is available outside the bone to receive attachment of a wire or a nut or other fastener used to attach a rod, wire, another appliance, or tissue such as a ligament or tendon to the bone at the location of the fixation device.

The foregoing and other objectives, features, and advantages of the invention will be more readily understood upon consideration of the following detailed description of the preferred embodiments, taken in conjunction with the accompanying drawings.

### BRIEF DESCRIPTION OF THE DRAWINGS

FIG. 1 is an isometric view of a bone-engaging attachment device that is one exemplary embodiment of an aspect of the invention.

FIG. 2 is an isometric view of a portion of an exterior surface of a bone, with a unicortical drill being used to prepare a hole in the bone to receive the attachment device shown in FIG. 1.

FIG. 3 is an isometric view of the portion of an exterior surface of a bone shown in FIG. 2, and showing a lateral channel.

FIG. 4 is an isometric view of an attachment device such as the one shown in FIG. 1 being placed into the hole shown in FIG. 2.

FIG. 5 is an isometric view of the attachment device shown in FIGS. 1 and 4 being moved into the lateral channel.

FIG. 6 is an isometric view showing the attachment device in place in the bone shown in FIGS. 2-5 with a nut mated with the threads on the shaft of the device to secure a wire to the bone in which the device is implanted.

FIG. 7 is a sectional view taken along line 7-7 of FIG. 6, showing a wire captured in a grooved washer.

FIG. 7A is a view similar to FIG. 7, showing a plate secured to a bone by the attachment device shown in FIGS. 1 and 5-7.

FIG. 7B is a view similar to FIG. 7, showing a rod fastened to a bone by the attachment device.

FIG. 8 is a sectional side elevational view of a human skull, showing an attachment device such as that shown in FIG. 1 implanted in the occipital region of the skull.

FIG. 9 is a posterior view of a portion of the skull shown in FIG. 8, showing two attachment devices such as that shown in FIG. 1 implanted in the occipital region.

FIG. 10 is a sectional, partially cut-away, and simplified view of a human shoulder joint, with an attachment device that is another exemplary embodiment of the present invention implanted in the shoulder joint.

### DETAILED DESCRIPTION OF THE PREFERRED EMBODIMENTS

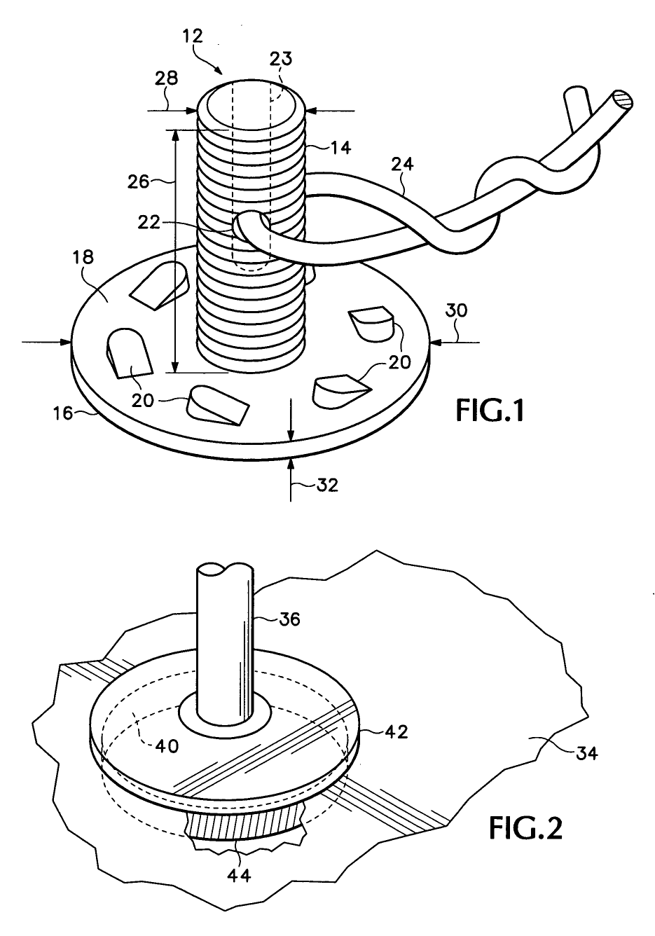

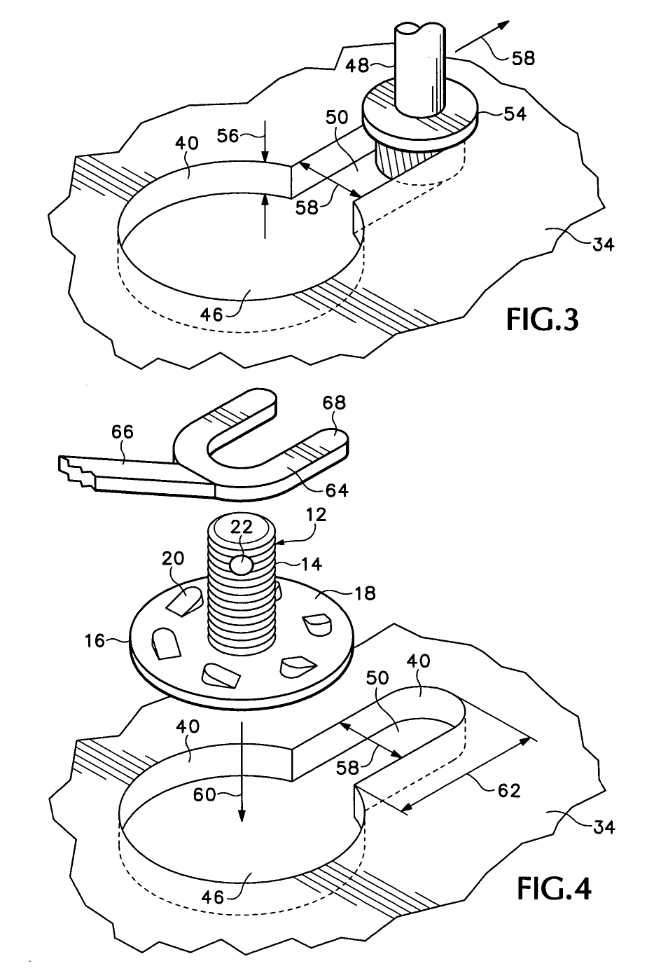

Referring now to FIGS. 1-9 of the drawings which form a part of the disclosure herein, a bone-engaging surgical fixation or attachment device 12 has a shaft 14 and a radially extending head 16 attached to one end of the shaft 14 with an engaging face 18 facing toward the shaft 14.

The shaft 14 of the device 12 may be threaded, as shown in FIG. 1, and a hole 22 may be provided, extending transversely through the shaft 6 and spaced apart from the engaging face 18 by a suitable distance, to make the shaft suitable to receive a connector, for example, a wire 24 to be fastened to a bone by use of the attachment device 12. Alternatively, the shaft might define a slot 23, shown in broken line, extending over at least a portion of its length, to receive a rod or wire that can be captured by a nut threaded onto the shaft 14.

Protruding from the engaging face 18 are several retainers 20, preferably evenly spaced, which, as shown in FIG. 1, may be inclined, wedge-shaped projecting bodies oriented around the shaft 14 where they can engage adjacent tissue so as to resist rotation of the device 12 in a direction which would tend to unscrew the shaft 14 from a threaded nut, as will be explained more fully presently.

The bone-engaging attachment device 12 may be made in any of a variety of sizes depending on the particular application, considering, for example, the size of the patient, the particular bone where its use is intended, and what wire, rod, or appliance is intended to be fastened to a bone. For example, for use in the occipital region of a human skull, the shaft 14 may have a length 26 of 8 mm, and a diameter 28 of 3 mm. The head 16 may have a diameter or maximum width 30 of 10 mm and a thickness 32 of 0.80 mm, and the device 12 may be manufactured of a suitable biologically compatible metal, such as titanium or surgical stainless steel.

The attachment device 12 is used by preparing a suitable hole in a bone where it is to be mounted. In the device 12, shown in FIG. 1 the head 16 is circular, and a corresponding circular hole may be prepared in a bone 34 by using a unicortical drill 36 to cut a circular hole to a depth 38 through the cortex 40 of the bone 34. A preferred unicortical drill 36 has a depth stop 42 projecting radially from its cutting portion 44 to limit the depth 38 of a hole 46 to a distance of, for example, 5 mm, no more than slightly deeper than the thickness of the cortex 40, although other drills or cutters could be used, taking care to limit the depth of the hole prepared.

Once the hole 46 is prepared using the unicortical drill 36, a relatively small side-cutting unicortical drill 48 is used to cut a channel 50, preferably extending radially outward from the hole 46, as indicated by the arrow 52. The side-cutting unicortical drill 48 also has a radially extending depth stop 54, so that the drill 48 can be used to cut the channel 50 with a depth 56 similar to the depth of the hole 46. The side-cutting unicortical drill 48 preferably has a diameter appropriate to cut the channel 50 with a width 58 equal to the diameter 28 of the shaft 14 of the attachment device 12 intended to be attached to the bone 34.

While the head 16 is shown herein as circular, other shapes could also be used, as where an elongated oval shape might be able to fit better within a bone at a particular location. A correspondingly shaped hole 46 of an appropriate size could then be prepared by use of an appropriate drill, perhaps drilling overlapping holes.

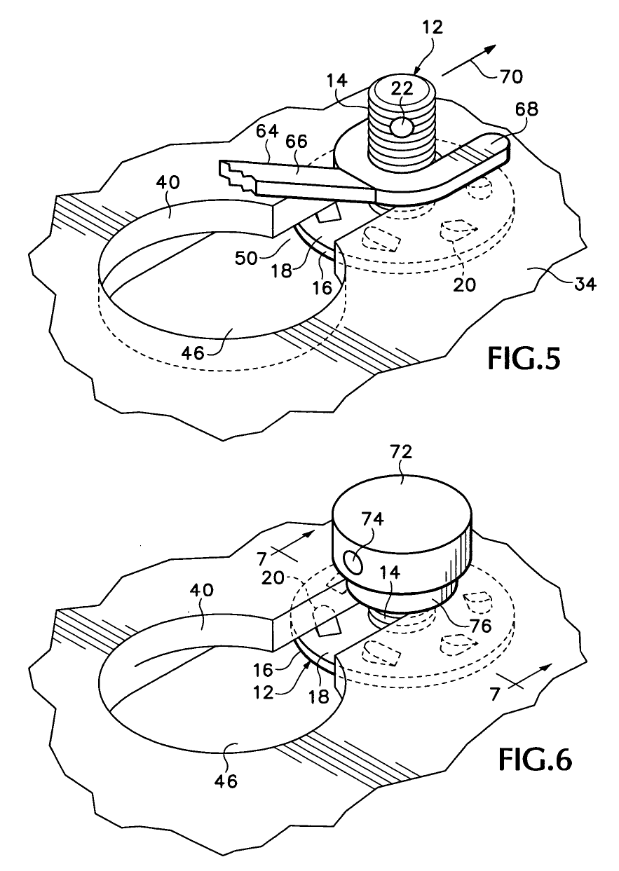

Referring next to FIGS. 4 and 5, the attachment device 12 is installed in the bone 34 by pressing it into the hole 46 as indicated by the arrow 60 in FIG. 4, so that the engaging face 18 of the head 16 is aligned slightly inward of the interior boundary of the cortex 40, resting in the cancellous tissue of the bone 34. The entire attachment device 12 is then moved laterally from the hole 46 into the channel 50. The length 62 of the channel 50, shown in FIG. 4, is great enough so that portions of the cortex 40 are adjacent the engaging face 18 except in the portion of the channel 50 between the shaft 14 and the hole 46, when the attachment device 12 is moved entirely into the channel 50, as shown in FIG. 5. The length 62 is thus preferably at least equal to the diameter 30 of the head 16, so that when the attachment device 12 has been moved the engagement face 18 is adjacent an interior boundary of the cortex 40 and the head 16 is prevented from being pulled outward from the bone 34 by its engagement with the cortex 40.

A tool 64, including a handle 66 shown partially cut away and a fork 68 of a size to fit the shaft 14, may be used conveniently to push the attachment device 12 in the direction of the arrow 70 shown in FIG. 5, from its initial position within the hole 46 to its desired final position in the channel 50. The thickness 32 of the head 16 is preferably no greater than is required for suitable strength, in order to minimize the force necessary to be exerted to push the head 16 through the cancellous tissue beneath the cortex 40 on either side of and at the end of the channel 50.

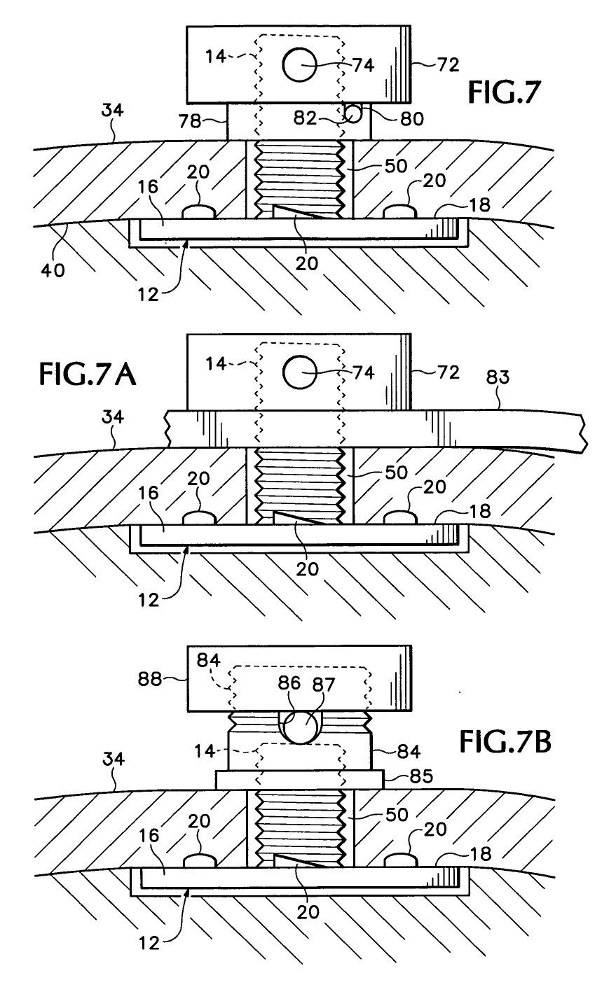

As shown in FIGS. 6, 7, 7A, and 7B, once the attachment device 12 has been placed in the channel 50 as shown in FIG. 5, various options are available, such as the installation of a nut 72 by threading it onto the shaft 14. The nut 72 may define a through-bore 74 useful to accept a spanner to tighten the nut 74 onto the shaft 14, and the through-bore 74 may thereafter be used to receive and hold a wire or other appliance to be attached to the bone 34 by means of the device 12. A washer 76 of the appropriate thickness is preferably installed on the shaft 14, between the nut 72 and the outer surface of the bone 34.

As the nut 72 is tightened onto the shaft 14 the head 16 is pulled upward against the inner face of the cortex layer 40, driving the retainers 20 into the tissue of the cortex 40, where they engage the relatively hard bone tissue of the cortex 40 to resist rotation of the head 16 as the nut 72 is tightened onto the shaft 14, as may be understood better with reference to FIG. 7. The retainers 20 may have other forms, but are preferably oriented so as to favor movement that would tend to tighten a threaded attachment and resist rotation in a loosening direction.

A similar but slightly different use of the attachment device 12 is shown in FIG. 7, wherein a special washer 78 is located between the nut 72 and the surface of the bone 34. The washer 78 includes a groove 80 in which a wire 82 is received and captured by the nut 72 as the nut 72 is tightened down upon the shaft 14 of the attachment device 12.

As shown in FIG. 7A, the nut 72 may be tightened onto the shaft 14 of the attachment device 12 to attach a plate 83 to a bone, as may be desired in rigidly connecting one bone to another by the plate 83.

As shown in FIG. 7B, a saddle nut 84 is threaded onto the shaft 14 above a washer 85 of a desired thickness to leave a saddle or slot 86 defined by the saddle nut available to receive a rod 87 which is captured in the slot 86 by a cap nut 88 threaded onto external threads on the saddle nut 84.

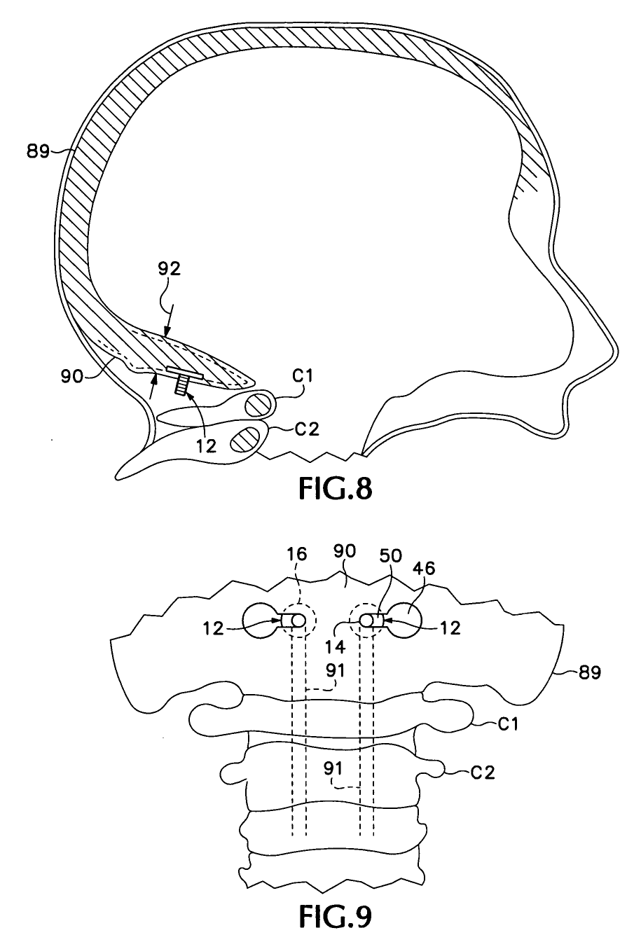

In FIGS. 8 and 9 one particular application of the attachment device 12 is illustrated. In connection with stabilization of fractured cervical vertebrae C1 and C2, it is desired in some cases to fuse vertebrae C1 and C2 together with each other and to the occipital portion 90 of the skull 89. To accomplish satisfactory stabilization of the skull 89 a pair of attachment devices 12 may be inserted in respective holes 46 and channels 50 in the occipital portion 90 of the skull 89, where the thickness 92 of the bone is ample for use of the device 12 and there is a great need for resistance to withdrawal of a fixation device because of the anticipated stresses related to the use of rods 91, shown in broken line in FIG. 9, for example, to stabilize the skull and the adjacent vertebrae C1 and C2.

Depending upon the location of the required attachment to a bone, the head 16 of an attachment device 12 may be flat, as shown in FIGS. 1-7, or the head 16 may be arcuately curved with an appropriate radius of curvature as to form a portion of a sphere, cylinder or cone, so that the engaging face 18 may be seated more fully in contact with the interior surface of the cortex of the bone where it is to be used.

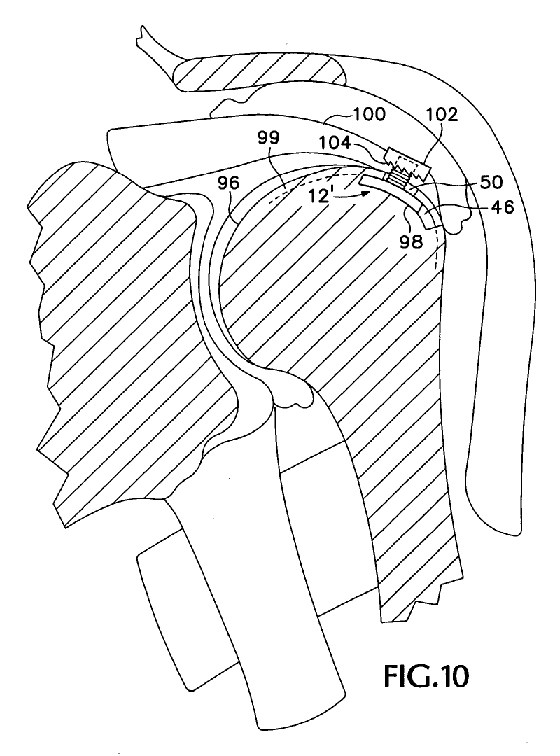

Thus, as shown in FIG. 10, an attachment device 12′ is shown mounted in the head 94 of a humerus 96 to provide a point of attachment of the supra-spinatus muscle M to the humerus 96 to repair a shoulder joint injury. The head 98 of the attachment device 12′ is curved to fit against the interior face of the cortex 99 of the head 98 of the humerus 96. The torn tendon 100 is attached to the attachment device 12′ by a tendon nut 102, whose surface facing toward the head 98 of the humerus is preferably equipped with slanted spikes or teeth 104 in order to more securely hold the tendon and resist loosening rotation of the nut 102 on the shaft 14 of the attachment device 12′ while the tendon 100 heals and reattaches itself to the bone tissue.

The terms and expressions which have been employed in the foregoing specification are used therein as terms of description and not of limitation, and there is no intention, in the use of such terms and expressions, of excluding equivalents of the features shown and described or portions thereof, it being recognized that the scope of the invention is defined and limited only by the claims which follow.

What is claimed is:

1. A bone-engaging attachment device, comprising:
(a) an elongate shaft having opposite ends;
(b) a head mounted on and extending radially from one of said ends of said shaft, said head having a width and a thickness significantly less than said width, and also having an engaging face directed toward said shaft.

2. The device of claim 1 wherein at least a portion of said shaft is threaded.

3. The device of claim 2 including a threaded tendon-fixing nut mated with said shaft.

4. The device of claim 1 including at least one retainer protruding from said engaging face.

5. The device of claim 1 including a plurality of inclined retainers protruding from said engaging face and oriented to resist rotation of said device about said shaft.

6. The device of claim 1 wherein said engaging face is shaped to fit against an inner surface of a cortical portion of a bone.

7. The device of claim 1 wherein said head is generally planar.

8. The device of claim 1 wherein said engaging face is generally convex and suitable for seating against a concave surface.

9. The device of claim 1, said shaft including a transverse through-bore spaced apart from said head.

10. The device of claim 1, said shaft including a transverse slot for receiving a rod.

11. A method of fastening an object to a bone, comprising:
(a) providing a bone-engaging attachment device having a shaft and a head located at an end of the shaft and having an engaging face directed toward the shaft;
(b) creating an opening in said bone extending through a cortex layer of said bone;
(c) creating a channel in said bone extending through said cortex layer and extending laterally from said opening, said channel being narrower than said opening;
(d) engaging said device with said bone by:
(i) inserting said head into said opening; and
(ii) thereafter moving said bone-engaging attachment device to a position wherein said head is adjacent an inner boundary of said cortex layer and the shaft extends outward through the channel, and wherein the device is thereby inhibited from becoming disengaged with said bone.

12. The method of claim 11 wherein said shaft is threaded.

13. The method of claim 11 including providing at least one retainer protruding from said engaging face and engaging said retainer in said cortex layer adjacent to said channel.

14. The method of claim 13 wherein said at least one retainer includes a plurality of retainers oriented to resist rotation of said attachment device.

15. The method of claim 11, including the step of seating said engaging face against an inner surface of said cortex layer of said bone.

16. The method of claim 11, wherein said step of engaging said device with said bone includes, after said step of inserting said head into said opening, pushing said device laterally until said shaft is located in said channel.

17. The method of claim 11, wherein said head is generally planar.

18. The method of claim 11, wherein said opening has a width, and said head has a width, and said width of said opening is substantially equal to said width of said head.

19. The method of claim 11, wherein said shaft has a thickness and said channel has a width, and said width of said channel is substantially equal to said thickness of said shaft.

20. The method of claim 9, wherein said engaging face is convex and suitable for seating against a concave surface.
